(19) United States
(12) Patent Application Publication
(10) Pub. No.: US 2015/0164953 A1
(43) Pub. Date: Jun. 18, 2015

(54) COMPOSITIONS AND METHODS OF TREATMENT WITH STEM CELLS

(71) Applicants: Keith Leonard March, Carmel, IN (US); Carmella Evans Molina, Zionsville, IN (US)

(72) Inventors: Keith Leonard March, Carmel, IN (US); Carmella Evans Molina, Zionsville, IN (US)

(21) Appl. No.: 14/565,081

(22) Filed: Dec. 9, 2014

Related U.S. Application Data
(63) Continuation of application No. 13/642,234, filed on Jan. 7, 2013, now abandoned.

(51) Int. Cl. A61K 35/28; A61K 35/44; A61K 38/18; A61K 35/39

(52) U.S. Cl. CPC A61K 35/28; A61K 35/39; A61K 35/44; A61K 38/1866

(57) ABSTRACT

The disclosure of the present application provides compositions and methods of treatment with stem cells. In at least one embodiment of a method for treating a patient with an insulin-related disorder, the method comprises the step of administering a cell-based composition to a patient with an insulin-related disorder to treat the insulin-related disorder, the cell-based composition comprising at least one mammalian stem cell and optionally at least one islet cell, the at least one mammalian stem cell capable of prolonging an effective life of the at least one islet cell.

### PRIORITY

The present U.S. continuation application is related to, and claims the priority benefit of, U.S. patent application Ser. No. 13/642,234, filed Jan. 7, 2013, which is related to, and claims the priority benefit of, PCT Patent Application Serial No. PCT/US11/33321, filed Apr. 20, 2011, which is related to, and claims the priority benefit of, U.S. Provisional Patent Application Ser. No. 61/326,002, filed Apr. 20, 2010, the contents of which are hereby incorporated by reference in their entirety into this disclosure.

### BACKGROUND

The discovery of pluripotent cells in the adipose tissue has revealed a novel source of cells that may be used for autologous cell therapy to regenerate tissue. The pluripotent cells reside in the “stromal” or “non-adipocyte” fraction of the adipose tissue; they were previously considered to be pre-adipocytes, i.e. adipocyte progenitor cells, however recent data suggest a much wider differentiation potential. Zuk et al. were able to establish differentiation of such subcutaneous human adipose stromal cells, or adipose stem cell cells as referred herein, (“ASCs”) in vitro into adipocytes, chondrocytes and myocytes. These findings were extended in a study by Erickson et al., which showed that human ASCs could differentiate in vivo into chondrocytes following transplantation into immune-deficient mice. More recently, it was demonstrated that human ASCs were able to differentiate into neuronal cells, osteoblasts cardiomyocyte, and endothelial cells.

EPCs (with a range of phenotypic definitions) have been studied extensively over the past decade since their original isolation from adult peripheral blood and, later from bone marrow, umbilical cord blood, vessel wall. Umbilical cord blood contains a population of EPC with a particularly high proliferative potential, termed endothelial colony forming cells (“ECFCs”). Recently, it was shown that ECFCs immobilized in matrices form functional vessels in vivo when implanted in mice. While the presence of blood cells within the capillary networks formed by such human EPCs confirmed anastomoses with host vasculature, the neo-vessels were limited in frequency and size. This finding is similar to a prior study with implants containing fully mature endothelial cells (“ECs”), in which vessels ere narrow-caliber and comprised of a single layer of cells. In the latter study, large caliber vessels with thick walls were formed only with ECs overexpressing the bcl-2 oncogene, presumably as a consequence of repressed EC apoptosis as well as augmented recruitment of host mesenchymal cells. With non-transformed ECs, the failure to establish stable, mature vasculature may be due to prolonged absence of a stabilizing layer of mural cells, which include pericytes and smooth muscle cells (“SMCs”). Although EPCs secrete multiple angiogenic factors to attract perivascular cells, conditions created in the matrix implants in vivo may restrict recruitment and, thereby, fail to prevent disassembly of vessels due to EC apoptosis. It has been demonstrated that human saphenous vein and aortic smooth muscle cells, blood derived and bone marrow MSC cooperate with ECs to promote stable vascular networks. However, the utility of these findings is restricted by the scarcity of adequate and easily-accessible sources of these perivascular/mural cell types.

Diabetes mellitus is a highly prevalent disease, afflicting more than 10% of the US population greater than 20 years of age; and more than 23% of the population greater than 60 years old (NIH-NIDDK statistics from 2007). Type 1 diabetes mellitus (TIDM) accounts for about 10% of diabetes, and results from a cascade of events that culminates in destruction of the insulin-producing 3 cells of the islets of Langerhans. These events are initiated after a nonspecific injury to the β cell results in exposure of autoantigens, after which macrophages and other antigen presenting cells activate CD4+ and CD8+ T cells. A complex and destructive interplay ensues, which is amplified by the secretion of proinflammatory cytokines such as interleukin 1β (IL-1β), tumor necrosis factor α (TNFα), and interferon γ (IFN γ) from macrophages, T cells, and the β cell itself, enabling “a vicious cycle” of necrotic and apoptotic β cell death.

Type 2 diabetes mellitus (T2DM) results from a combination of peripheral insulin resistance and β-cell dysfunction. In the initial phases of disease, the β cell is able to compensate for the insulin resistance by increasing insulin production, resulting in hyperinsulinemia. However, this compensation is limited in time; as the β cell function begins to fail as a result of increasing metabolic demands, the insulin levels fall. The United Kingdom Prospective Diabetes Study demonstrated that patients with T2DM experience progressive β cell dysfunction despite most drug treatments to lower blood glucose; this dysfunction is characterized by profound insulin secretory defects. Clinically, this manifests as a loss of the first-phase response to intravenous glucose, delayed and blunted insulin responses to ingestion of a mixed meal, and loss of the normal pattern of insulin secretion. In T2DM, the fluid milieu of the body typically exhibits hyperglycemia (again, expressed over time in terms of HbA1c) in the context of hyperinsulinemia.

While there are important differences in the underlying pathophysiology of the two forms of diabetes, β cell failure remains at the core of both Type 1 and Type 2 diabetes. Likewise, treatments that successfully employ β cell replacement could have utility in TIDM and T2DM.

In 2000, Shapiro et al. published a highly promising account of islet transplantation at the University of Alberta (Edmonton, Canada), where 7 out of 7 patients with TIDM who were treated with islet transplantation remained insulin-independent after one year. Tremendous interest in advancing the field of islet transplantation ensued. However to date, long term results from islet transplants have been somewhat disappointing, largely due to islet graft failure. Several reasons for the graft failure have been proposed, including inadequate graft mass due to acute inflammatory destruction; problems with islet quality, viability, and engraftment; auto and allo-immune destruction; and inadequate or abnormal revascularization of the islet graft. The normal islet is highly vascular with approximately 10 times more blood delivered to the endocrine pancreas as compared to exocrine tissue. This is an impressive discrepancy given the consideration that endocrine cells comprise only 1-2% of the total mass of the pancreas. Normally, a central arteriole supplies blood to the islet through a highly fenestrated capillary network, and this native vascular system is disrupted during the isolation procedure.

A treatment capable of increasing the efficacy of islet cell transplantation, or otherwise promoting glucose homeostasis in a mammal would be greatly appreciated.

### BRIEF DESCRIPTION

Disclosed herein are various methods and compositions for treating a patient having a disorder. At least some of the methods and compositions involve the use of mammalian stem cells, such as mammalian adipose stem cells, to treat the disorder.

In at least one embodiment of a method for treating a patient with a disorder, such as an insulin-related disorder, the method comprises the step of administering a cell-based composition to a patient with an insulin-related disorder to treat the insulin-related disorder, the cell-based composition comprising at least one mammalian stem cell and optionally at least one islet cell, the at least one mammalian stem cell capable of prolonging an effective life of the at least one islet cell. The step of administering a cell-based composition, in at least one embodiment of the method, comprises administering the cell-based composition comprising at least one mammalian adipose stem cell, and optionally at least one mammalian endothelial or endothelial progenitor stem cell. Further, the at least one mammalian stem cell of the cell-based composition administered to the patient may previously have been isolated from the patient. Moreover, administering the cell-based composition to a patient comprise a method of administration selected from the group consisting of intravenous injection, intramuscular injection, subcutaneous injection, retrograde venous injection, arterial injection, and surgical implantation.

In at least one embodiment of a method for treating a patient with an insulin-related disorder, the insulin-related disorder is selected from the group consisting of Type 1 diabetes, Type 2 diabetes, gestational diabetes, pre-diabetes, and impaired glucose tolerance. Additionally, the cell-based composition may further comprise at least one islet cell and/or a biological agent capable of promoting cell growth. The biological agent may comprise a growth factor, including but not limited to a hepatocyte growth factor, an insulin-like growth factor, a fibroblast growth factor, and a vascular endothelial growth factor. The biological agent may also be an anti-apoptotic agent or a pro-angiogenic agent. Additionally, in at least one embodiment of the method, the cell-based composition is provided in a form selected from the group consisting of a matrix form and a capsule form.

The administration step, in at least one embodiment of the method of treating a patient, is performed to treat an insulin-based disorder by promoting production of insulin within the patient. Further, the administration step may be performed to treat the insulin-based disorder by at least one of (1) reducing a rate of peripheral insulin resistance within the patient, (2) reducing a rate of β-cell dysfunction within the patient, and (3) increasing the patient's glucose tolerance.

In at least one embodiment of the method or composition of the present disclosure, the at least one mammalian stem cell is selected from the group consisting of at least one a CD10+ mammalian adipose stem cell, at least one a CD13+ mammalian adipose stem cell, at least one a CD34+ mammalian adipose stem cell, at least one a CD34− mammalian adipose stem cell, at least one a CD45+ mammalian adipose stem cell, at least one a CD45-mammalian adipose stem cell, at least one a CD90+ mammalian adipose stem cell, at least one a CD90− mammalian adipose stem cell, at least one a CD140a+ mammalian adipose stem cell, at least one a CD140a− mammalian adipose stem cell, at least one a CD140b+ mammalian adipose stem cell, and at least one a CD140b− mammalian adipose stem cell.

In at least one embodiment of a method for treating a patient with a disorder, the cell-based composition further comprises at least one endothelial cell. Additionally, in at least one embodiment, the ratio of the at least one mammalian stem cell to the at least one endothelial cell is selected from the group consisting of at least about 8 to about 1, about 4 to about 1, about 2 to about 1, about 1 to about 1, about 1 to about 2, about 1 to about 4, about 1 to at least about 8.

In at least one embodiment of a method for treating a patient with an insulin-related disorder, the method comprises the step of administering a cell-based composition to a patient with an insulin-related disorder to treat the insulin-related disorder, the cell-based composition comprising at least one mammalian stem cell and at least one islet cell, the at least one mammalian stem cell capable of prolonging an effective life of the at least one islet cell and further capable of effectuating promotion of insulin production within the patient. Further, the cell-based composition may also comprises a biological agent capable of promoting cell growth, the biological agent selected from the group consisting of a hepatocyte growth factor, an insulin-like growth factor, a fibroblast growth factor, a vascular endothelial growth factor, an anti-apoptotic agent and a pro-angiogenic agent.

In at least one embodiment of a method for treating a patient with a disorder, the method comprises the step of administering a cell-based composition to a patient with a disorder to treat the disorder, the cell-based composition comprising at least one mammalian stem cell and optionally at least one islet cell, the at least one mammalian stem cell capable of prolonging an effective life of the at least one islet cell. The step of administering a cell-based composition in at least one embodiment of the method of treatment comprises administering the cell-based composition to a patient with an insulin-related disorder, where the insulin-related disorder may be selected from the group consisting of Type 1 diabetes, Type 2 diabetes, gestational diabetes, pre-diabetes, and impaired glucose tolerance.

In at least one embodiment of a method for treating a patient with an insulin-related disorder, the method comprises the step of administering a cell-based composition to a patient with an insulin-related disorder to treat the insulin-related disorder, the cell-based composition comprising at least one mammalian stem cell capable of effectuating promotion of insulin production within the patient.

In at least one embodiment of a cell-based composition of the present disclosure, the cell-based composition comprises at least one mammalian stem cell, and optionally at least one islet cell, wherein the at least one mammalian stem cell capable of prolonging an effective life of the at least one islet cell, and wherein the composition is effective to treat a patient with an insulin-related disorder by effectuating the promotion of insulin production within the patient. Optionally, the cell based composition may further comprise at least one islet cell and/or a biological agent capable of promoting cell growth. The biological agent may comprise a growth factor, including but not limited to a hepatocyte growth factor, an insulin-like growth factor, a fibroblast growth factor, and a vascular endothelial growth factor. Further, the biological agent may be selected from the group consisting of an anti-apoptotic agent and a pro-angiogenic agent.

In at least one embodiment of the cell-based composition, the cell-based composition is provided in a form selected from the group consisting of a matrix form and a capsule form. Additionally, the cell-based composition may further comprise a biologically-compatible carrier. Further, the cell-based composition may be effective to treat the patient by at least one of (1) reducing a rate of peripheral insulin resistance within the patient, (2) reducing a rate of β-cell dysfunction within the patient, (3) increasing the patient's glucose tolerance. In at least one embodiment of the composition, the at least one mammalian stem cell comprises at least one mammalian adipose stem cell.

In at least one embodiment of a cell-based composition of the present disclosure, the cell-based composition comprises at least one islet cell, and at least one mammalian stem cell capable of prolonging an effective life of the at least one islet cell, wherein the composition is effective to treat a patient with an insulin-related disorder by effectuating the promotion of insulin production within the patient. Additionally, an embodiment of the cell-based composition may further comprise a biological agent capable of promoting cell growth, the biological agent selected from the group consisting of a hepatocyte growth factor, an insulin-like growth factor, a fibroblast growth factor, a vascular endothelial growth factor, an anti-apoptotic agent and a pro-angiogenic agent.

In at least one embodiment of a method of producing a cell-based composition useful to treat a patient, the method comprises the steps of isolating at least one mammalian stem cell from a mammal, optionally expanding the at least one mammalian stem cell to produce a plurality of mammalian stem cells, and combining at least some of the plurality of mammalian stem cells or the isolated at least one mammalian stem cell with at least one islet cell to form a cell-based composition effective to treat a disorder of a patient. The at least one mammalian stem cell may in at least one embodiment comprise at least one mammalian adipose stem cell. Additionally, an embodiment of the method of producing a cell-based composition may further comprise the step of administering the cell-based composition to the patient to treat the disorder, including but not limited to Type 1 diabetes, Type 2 diabetes, gestational diabetes, pre-diabetes, and impaired glucose tolerance. Further, the mammal in an embodiment of the method of producing a cell-based composition may be the patient. Additionally, the step of expanding may comprise expanding the at least one mammalian stem cell in a cell culture environment to produce the plurality of mammalian stem cells.

In at least one embodiment of a method of vascularizing tissue, the method comprises the steps of combining at least one mammalian stem cell with a plurality of endothelial cells and a matrix to create a vascularization composition, and administering the vascularization composition to a patient, wherein the vascularization composition is useful to increase vessel formation at the site of administration. The at least one mammalian cell of an embodiment of a method or composition of the present disclosure may be VEGF⁺ and HCF⁺, and may further be from the patient. Further, the at least one mammalian stem cell comprises at least one adipose stem cell.

In at least one embodiment of a method of treating a patient, the step of administering effectuates vascularization of a tissue of the patient at or near the site of administration of the cell-based composition.

In at least one embodiment of a method to determine the effectiveness of a cell-based composition to treat a mammalian disorder, the method comprises the steps of placing at least one mammalian stem cell in a first cell vessel, placing at least one islet cell in the first cell vessel with the at least one mammalian stem cell, placing an additional at least one mammalian stem cell in a second vessel that does not contain a mammalian stem cell, and comparing a selective morbidity of the at least one islet cell in the first vessel and the second vessel, and wherein the comparison is indicative of an ability of the at least one mammalian stem cell to prolong an effective life of the at least one islet cell in the first cell vessel, which is indicative of an effectiveness of the at least one mammalian stem cell to treat a mammalian disorder. Optionally, the step of comparing the selective morbidity may also comprise the step of determining the selective morbidity of the at least one islet cell in the first vessel and the second vessel with a diagnostic agent, such as an antibody, a reactive chemical compound, and a labeled molecule.

### BRIEF DESCRIPTION OF THE DRAWINGS

FIG. 1A shows a flowchart depicting the step for a method of treating a patient with an insulin related disorder, according to at least one embodiment of the present disclosure;

FIG. 1B shows a flowchart depicting the steps for vascularizing a tissue, according to at least one embodiment of the present disclosure;

FIG. 2A shows a flowchart depicting the steps for a method of producing a cell-based composition, according to at least one embodiment of the present disclosure;

FIG. 2B shows a flowchart depicting the steps for a method of determining the effectiveness of a cell-based composition to treat a mammalian disorder, according to at least one embodiment of the present disclosure;

FIGS. 3A and B show a flow cytometric analysis of adipose stem cells (ASCs) for co-expression of CD34 with mesenchymal (panel A) and pericyte markers (panel B), according to at least one embodiment of the present disclosure;

FIGS. 4a-h show the histological analysis of human adipose tissue, according to at least one embodiment of the present disclosure;

FIG. 5 shows a graphical representation of the influence of conditioned media from ASCs on human microvascular endothelial cell (HMVEC) survival and proliferation, according to at least one embodiment of the present disclosure;

FIG. 6 shows a visual representation of a growth factor and cytokine profile of ASCs, according to at least one embodiment of the present disclosure;

FIG. 7 is a microscopic view of a vascular network formed by endothelial cells (EC) plated on an established monolayer of ASCs, according to at least one embodiment of the present disclosure;

FIGS. 8a-e show visual (panels a-c), graphical (panel d), and histological (panel e) depictions of the synergy between ASCs and ECs in promoting vasculogenesis, according to at least one embodiment of the present disclosure;

FIG. 9 shows an intravital microscopic view of a kidney from a living mouse, according to at least one embodiment of the present disclosure;

FIG. 10 shows a graphical representation of vessel formation of ASC combined with varying endothelial cell types, according to at least one embodiment of the present disclosure;

FIGS. 11A-C show the visual effects (panels A and B) and graphical (panel C) depictions of effects of ASC treatment on limb necrosis, according to at least one embodiment of the present disclosure;

FIGS. 12a-d show visual (panels a-c) and graphical (panel d) representations of the effect of ASC deficient in HGF on relative perfusion rates in ischemic limbs, according to at least one embodiment of the present disclosure;

FIGS. 13A-D show the graphical representations of expanded islet cell function, according to at least one embodiment of the present disclosure;

FIGS. 14A-C shows a graphical representation (panel A) of the effect of co-culturing islet cells with ASC on the Insulin Stimulatory Index, and microscopic views (panels B and C) showing the breakdown of the islet capsule, according to at least one embodiment of the present disclosure;

FIG. 15 shows the microscopic effects of co-culturing ASC with islet cells on survival of islet cells, according to at least one embodiment of the present disclosure;

FIG. 16 shows the graphical representation of restoration of normoglycemic state by heterotypic transplantation of pancreatic islets, according to at least one embodiment of the present disclosure;

FIGS. 17A-C show graphical representations of increased glucose tolerance due to treatment with ASCs (pre-treatment with ASC, panel A; 7 days post-treatment with ASC, panel B; and 25 days post-treatment with ASC, panel C), according to at least one embodiment of the present disclosure;

FIGS. 18A-C show the histological analysis of collagen matrices containing ASC, EC and porcine pancreatic islets after implantation for two weeks in NOD-SCID mice, according to at least one embodiment of the present disclosure;

FIG. 19 shows a graphical representation of the effect of ASC treatment on restoration of glucose hemostasis, according to at least one embodiment of the present disclosure;

FIG. 20 shows a graphical representation of VEGF secretion by ASC, according to at least one embodiment of the present disclosure;

FIG. 21 shows a graphical representation of blood glucose levels over time in mice inoculated with ASC, according to at least one embodiment of the present disclosure;

FIG. 22 shows an immunofluroesence analysis of islets stained to determine live and dead cells, according to at least one embodiment of the present disclosure;

FIG. 23 shows a graphical representation of dead cells in an islet from an STZ treated mouse which had been inoculated with (or without) ASC, according to at least one embodiment of the present disclosure;

FIG. 24 shows a histological analysis of the pancreas and lung to visualize insulin producing regions, according to at least one embodiment of the present disclosure;

FIGS. 25A-C show a graphical representation of blood glucose levels in control mice, diabetic mice (STZ), and diabetic mice inoculated with ASC (STZ+ASC) before inoculation with ASC (panel A), at 7 days following ASC inoculation (panel B), and 25 days following ASC inoculation (panel C), according to at least one embodiment of the present disclosure;

FIG. 26 shows a graphical representation of serum insulin levels in mice at 7 days post inoculation with ASC, according to at least one embodiment of the present disclosure;

FIG. 27 shows a histological analysis of pancreata stained with insulin-specific antibody to visualize insulin, according to at least one embodiment of the present disclosure;

FIG. 28 shows a graphical representation of Beta cell mass in mg in pancreata for control, diabetic (STZ), or diabetic and ASC treated (STZ+ASC) mice, according to at least one embodiment of the present disclosure;

FIG. 29 shows a histological analysis of islet cells with anti-PHS, according to at least one embodiment of the present disclosure; and

FIG. 30 shows a graphical representation of PH3 expression in control, diabetic (STZ), or diabetic and ASC treated (STZ+ASC) mice, according to at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The disclosure of the present application provides various methods for cell-based therapies. For instance, at least some embodiments of the methods disclosed herein heighten the treatment potential of islet cells. Due to the failure of islet cell grafts for the treatment of Type I and Type II diabetes, and considering the increasing level of diabetes among the general population, there is need for a method of treating diabetes.

Adipose stem cells (ASCs) are isolated from human, and other mammalian, subcutaneous adipose tissue according to the method of Zuk et al. ASCs are predominantly localized in the peri-endothelial layer of the vessels in vivo (in adipose tissue), and are phenotypically and functionally equivalent to pericytes associated with microvessels. The ASCs may, in at least one illustrative example, be isolated at a level of about 10⁸ cells per 100 ml of lipoaspirate. Further, following isolation, the isolated ASCs may be cultured on tissue culture plastic in EGM-2mv medium. In this medium, ASC can expand to about 1000-fold over a 10 day period. Further, ASCs isolated from humans (hASCs) routinely secrete a wide variety of bioactive molecules, such as VEGF, HGF, and GM-CSF, which participate in stimulation of EC survival and proliferation and stabilization of endothelial networks formed on the surface of Matrigel.

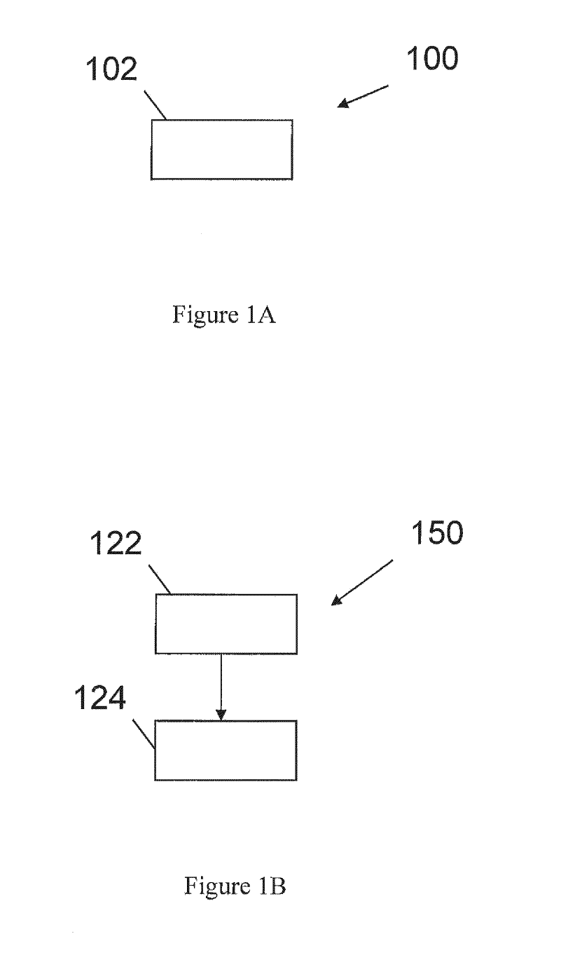

Referring to FIG. 1A, at least one embodiment of a method 100 of treating a patient with a disorder is depicted. Exemplary method 100 comprises the step of administering a cell-based composition to a patient with an insulin-related disorder to treat the insulin-related disorder, the cell-based composition comprising at least one mammalian stem cell and optionally at least one islet cell, the at least one mammalian stem cell capable of prolonging an effective life of the at least one islet cell (exemplary administering step 102). “Islet cells” as used herein shall have the meaning of at least one cell from the Islet of Langerhans, or portion thereof, or at least one β cell. Optionally, an exemplary cell-based composition may further comprise at least one endothelial cell. Additionally, the ratio of mammalian stem cells (such as adipose stem cells) to endothelial cells, in an exemplary cell-based composition, is at least about 8 to about 1, or about 4 to about 1, or about 2 to about 1, or about 1 to about 1, or about 1 to about 2, or about 1 to about 4, or about 1 to at least about 8. In an exemplary embodiment, the at least one mammalian stem cell is originally isolated from the patient. Further, according to at least one embodiment, the step of administering the treated islet cell mixture may be performed by a route selected from a group consisting of intravenous injection, intramuscular injection, subcutaneous injection, retrograde venous injection, arterial injection, and surgical implantation.

According to an exemplary cell-based composition of the present disclosure, the composition further comprises a biological agent. The biological agent, in at least one exemplary embodiment, is selected from a group consisting of hepatocyte growth factor, insulin-like growth factor, fibroblast growth factor, vascular endothelial growth factor, an anti-apoptotic agent, and a pro-angiogenic agent. Treatment of the at least one mammalian stem cell with a biological agent may be for a period of at least about one minute, at least about twelve hours, at least about twenty-four hours, at least about forty-eight hours, or at least about 72 hours. Optionally, an embodiment of a cell-based composition may also comprise at least one endothelial cell. Moreover, an exemplary cell-based composition may further be provided in a form, such as a matrix form and a capsule form. The form, in at least one example, may comprise collagen, fibronectin, a combination thereof, or any acceptable and biocompatible form.

In at least one embodiment of the method 100 of treating a patient, the patient has an insulin-based disorder, such as Type 1 diabetes, Type II diabetes, or gestational diabetes. Further, the step of administering the cell-based composition to the patient treats the patient's insulin-based disorder.

According to at least one embodiment of an cell-based composition of the present disclosure, the composition comprises at least one islet cell, and at least one adipose stem cell, wherein the at least one adipose stem cell is capable of repressing cell death of the at least one islet cell. Optionally, the composition may further comprise at least one endothelial cell. Further, an cell-based composition may also comprise a biological agent selected from a group consisting of hepatocyte growth factor, insulin-like growth factor, fibroblast growth factor, vascular endothelial growth factor, an anti-apoptotic agent, and a pro-angiogenic agent.

According to at least one embodiment of the present disclosure, combining the at least one islet cell with at least one mammalian stem cell, that may be an adipose stem cell, and optionally at least one endothelial cell, generates a cell-based composition that is capable of at least one of (1) promoting the production of insulin within the patient when introduced, (2) reducing the rate of peripheral insulin resistance within the patient when introduced, (3) reducing the rate of β-cell dysfunction within the patient when introduced, and (4) increasing the patient's glucose tolerance when introduced. Additionally, in at least one embodiment, the administering of the cell-based composition to a patient increases vascular blood flow at the site of administration, and increases the level of blood glucose control of the patient.

Referring to FIG. 1B, at least one embodiment of a method 150 of vascularizing tissue is depicted. Exemplary method 150 comprises the steps of combining at least one mammalian stem cell with a plurality of endothelial cells and a matrix to create a vascularization composition (exemplary combining step 122) and administering the vascularization composition through any applicable method described herein to a patient (exemplary administering step 124). The at least one mammalian stem cell may be any embodiment described herein, and may additionally be VEGF⁺ and HGF⁺.

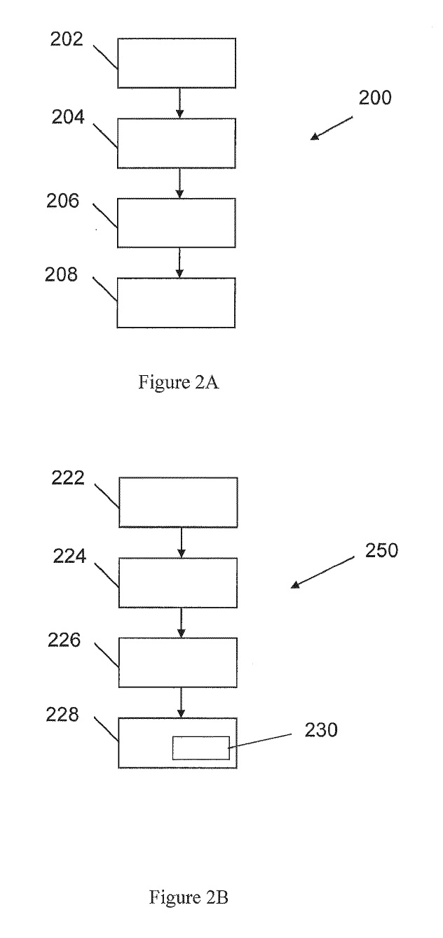

Referring to FIG. 2A, at least one embodiment of a method 200 of producing a cell-based composition useful to treat at patient is depicted. Exemplary method 200 comprises the steps of isolating at least mammalian stem cell from a mammal (exemplary isolating step 202), expanding the at least one mammalian stem cell to produce a plurality of mammalian stem cells (exemplary expanding step 204), combining at least some of the plurality of mammalian stem cells with at least one islet cell to form a cell-based composition effective to treat a disorder of a patient (exemplary combining step 206), and optionally administering an exemplary cell-based composition to the patient to treat the disorder (exemplary administering step 208). The at least one mammalian stem cell and the cell-based composition of method 200 may be any respective embodiment as described herein.

Referring to FIG. 2B, at least one embodiment of method 250 to determine the effectiveness of a cell-based composition to treat a mammalian disorder is depicted.

Exemplary method 250 comprises the steps of placing an embodiment of at least one mammalian stem cell in a first vessel (exemplary placing step 222), placing at least one islet cell in the first vessel with the at least one mammalian stem cell (exemplary placing step 224), placing an additional at least one mammalian stem cell in a second vessel that does not contain a mammalian stem cell (exemplary placing step 226), and comparing a selective morbidity of the at least one islet cell in the first vessel and the second vessel (exemplary comparing step 228). In at least one embodiment of method 250, comparing step 228 may further comprise the step of determining the selective morbidity of the at least one islet cell in the first vessel and the second vessel with a diagnostic agent (exemplary determining step 230). An exemplary diagnostic agent may include any compound, chemical, or biological component which may interact with a target or byproduct of a target. For example, an diagnostic agent may comprise an antibody, a reactive chemical compound, a labeled molecule, or any combination thereof. Antibodies used in an embodiment of the present disclosure may be monoclonal or polyclonal and derived from any species (e.g. human, rat, mouse, rabbit, pig). Further, indicator molecules may be aptamers, proteins, peptides, small organic molecules, natural compounds (e.g. steroids), non-peptide polymers, MHC multimers (including MHC-dextramers, MHC-tetramers, MHC-pentamers and other MHC-multimers), or any other molecules that specifically and efficiently bind to other molecules are also marker molecules.

Labeled molecules, for use as indicator compounds, may be any molecule that absorbs, excites, or modifies radiation, such as the absorption of light (e.g. dyes and chromophores) and the emission of light after excitation (fluorescence from flurochromes). Additionally, labeled molecules may have an enzymatic activity, by which it catalyzes a reaction between chemicals in the near environment of the labeling molecules, producing a signal which include production of light (chemi-luminescence) or precipitation of chromophors, dyes, or a precipitate that can be detected by an additional layer of detection molecules.

Exemplary fluorescence labels may produce the presence of light at a single wavelength, or a shift in wavelengths.

### EXAMPLES

### Example 1

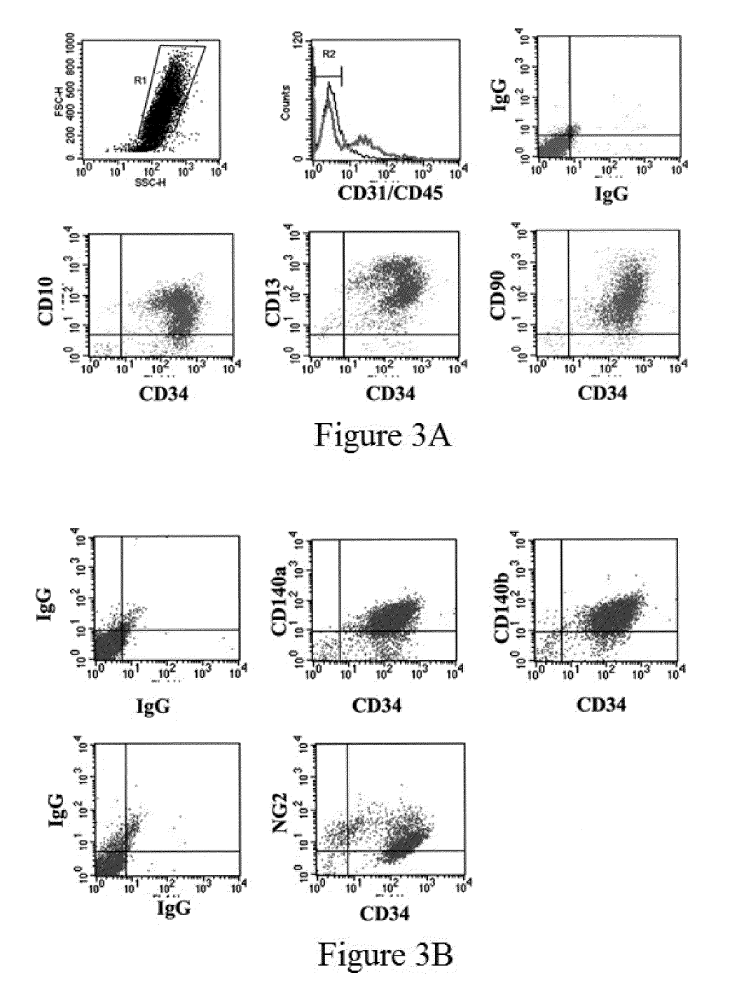

A majority of human ASCs (hASCs) isolated as described Zuk et al. and additionally enriched by attachment to tissue culture plastic, express the stem cell marker CD34 (in the first days of culture), as well as co-express several mesenchymal cell markers (CD10+/CD13+/CD90+) and pericyte markers (CD140a+/CD140b+/NG2+) (FIG. 3). Determination of these cell markers was conducted two days post-attachment to plastic by flow cytometric analysis of ASCs for co-expression of CD34 with mesenchymal (A) and pericyte markers (B). Analysis was performed for CD34 (APC), CD45 (FITC), CD10 (PE) and CD13 (PE) and CD90 (PE), CD140a (PE), and CD140b.

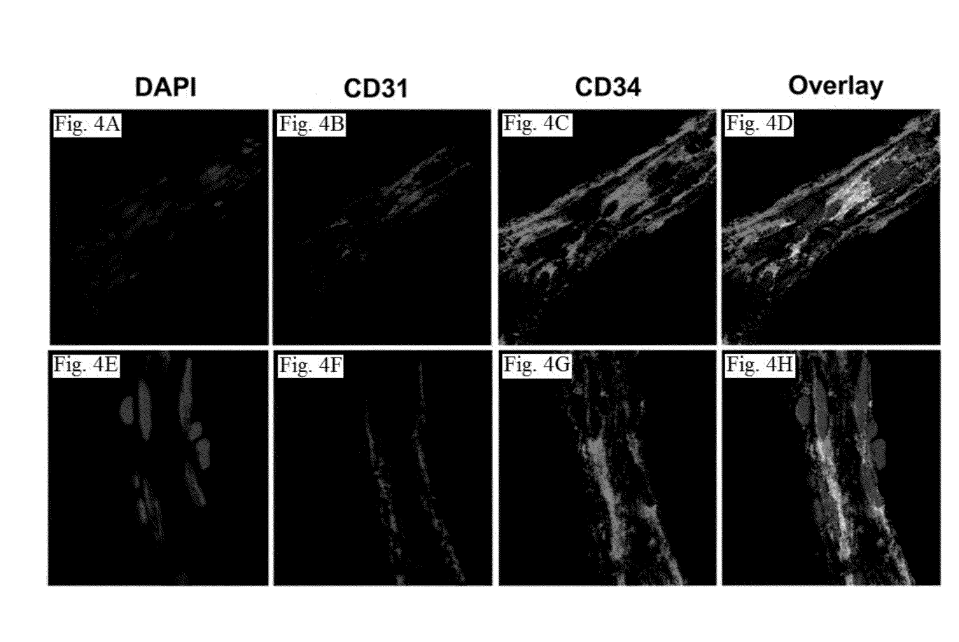

Following the identification of ASC markers, the location of ASC in adipose tissue was determined in situ by immunochemical staining. Staining for CD34 (FIG. 4) or CD140b (data not shown) revealed that ASC are located in a perivascular position, consistent with many ASC being mural blood vessel cells or pericytes. The histological analysis of human adipose tissue was conducted on frozen sections of human fat that were stained for the endothelial marker CD31 (red) and a major fresh ASC marker CD34 (green). Nuclei are revealed by 4,6-diamidino-2-phenylindole (DAPI) staining. Localization of ASC showed a close spatial relationship between ASC and endothelial cells (EC).

### Example 2

To address ASC-EC interactions, endothelial progenitor cells (EPCs) were isolated and expanded from umbilical cord vein blood of healthy newborns. Isolated mononuclear cells (MNC) were cultured on collagen-coated plastic in EGM-2/10% FBS. Cells were expanded and utilized up to passage 6, without significant changes in cell morphology, markers, and responses to factor stimuli). Throughout the work presented herein, EPCs derived from cord blood by this technique were used, to maximize consistency.

### Example 3

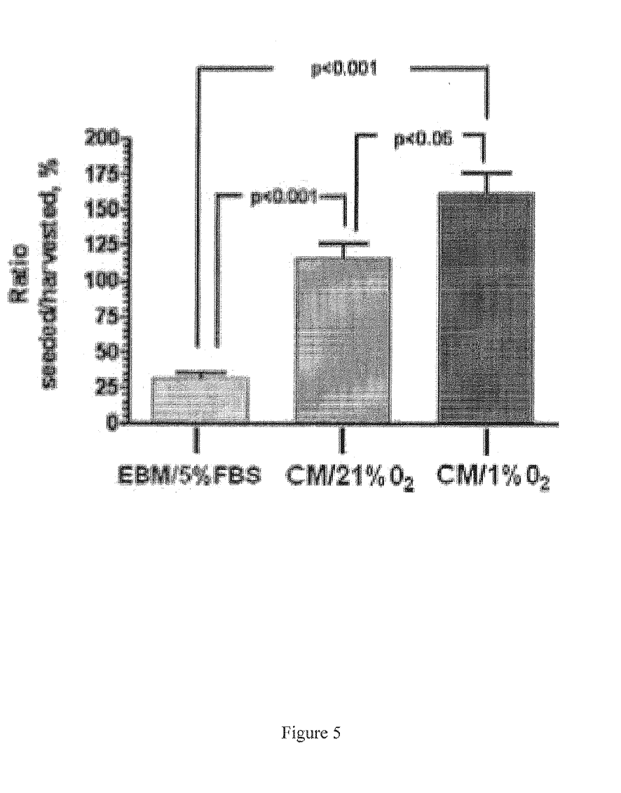

To evaluate the effect of factors secreted by hASCs on ECs, human microvascular ECs (HmVEC) cultured in growth factor-free media were exposed to conditioned media (CM) of hASCs incubated for 72 hours in either normoxic or hypoxic conditions (FIG. 5). A four day exposure of HmVEC to ASC-normoxic and ASC-hypoxic CM resulted in a marked increase in EC viability under conditions of limiting growth factors, with hypoxic medium demonstrating significantly increased activity. Conditioned media was generated from ASCs cultured in basal medium (EBM/5% FBS) at ambient oxygen (21%) or hypoxia (1%) conditions. The effect of hypoxia was accompanied by induction of both VEGF and HGF (data not shown), consistent with a hypoxia response, likely mediated by HIF-1α and HIF-2α.

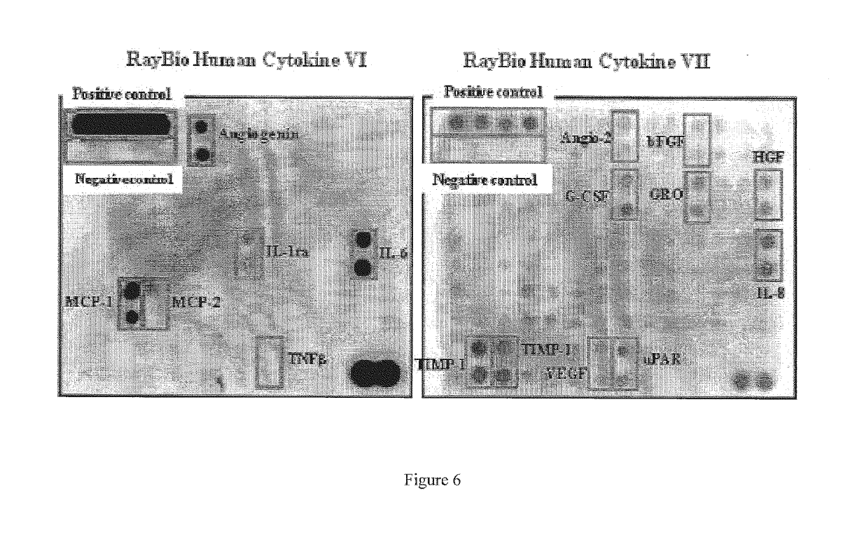

To understand a broader range of factors that could additionally participate in the effect on EC, ASC-normoxic CM (72 hours) was evaluated using RayBio Cytokine Antibody Arrays (RayBiotech Inc). FIG. 6 illustrates the secretion by ASC of multiple angiogenic factors (angiogenin, VEGF, HGF, bFGF and B-NGF); inflammatory factors (IL-6, -8, -11, -17, MCP-1, 2); and mobilizing factors (GM-CSF and M-CSF). Conditioned medium from 72 hour cultures in EBM-2/5% FBS were analyzed by antibody array membranes. Red frames denote angiogenesis that was significantly more abundant than in the control membranes probed with fresh media as a control (not shown).

### Example 4

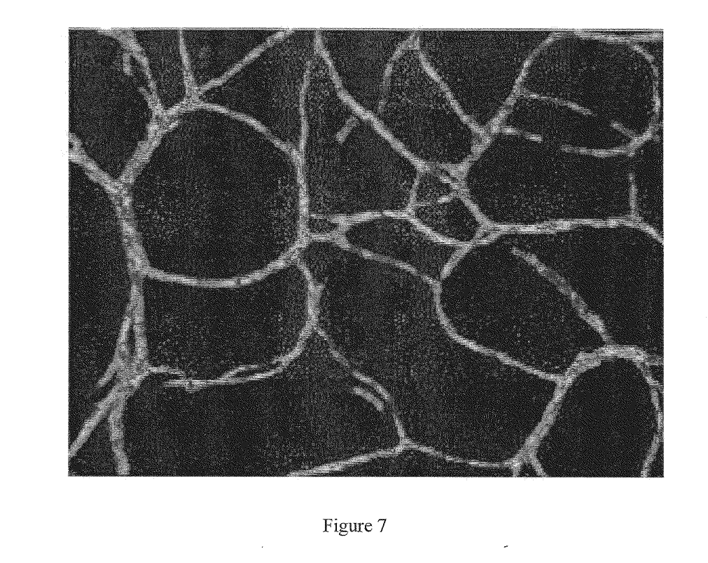

Culturing endothelial cells on tissue culture plastic alone or coated with extracellular matrix proteins results in cell expansion to a monolayer. To examine the functional interaction of ASC and EC, EC were plated on a monolayer of ASC directly on plastic, without the addition of exogenous ECM proteins. Co-culture under these conditions promotes spontaneous assembly of EC into vascular networks over 3-6 days (FIG. 7). Additionally, these networks in turn are stable for at least two weeks (duration of experiment). Through computational methods for quantitative characterization of such networks, the ASC monolayer was shown to have a significantly higher potential to support vascular network formation by EPC than either fibroblasts or smooth muscle cells (total tube length in ASC group—3.69±0.19 mm/mm², NHDF—1.14±0.23 mm/mm², coronary artery SMC—0.96±0.06 mm/mm², aortic SMC—1.66±0.08 mm/mm²). Parameters collected in this analysis included: total and average tube length, density of branchpoints and total area covered by the network. Studies to determine major factors involved in mediating ASC-driven assembly have demonstrated that two ASC-secreted factors, VEGF and HGF, contribute significantly to vascular network development (total tube length: control —5.7±0.12 mm/mm²; antiVEGF—2.2-0.4 mm/mm²; antiHGF—4.0±0.2 mm/mm²).

### Example 5

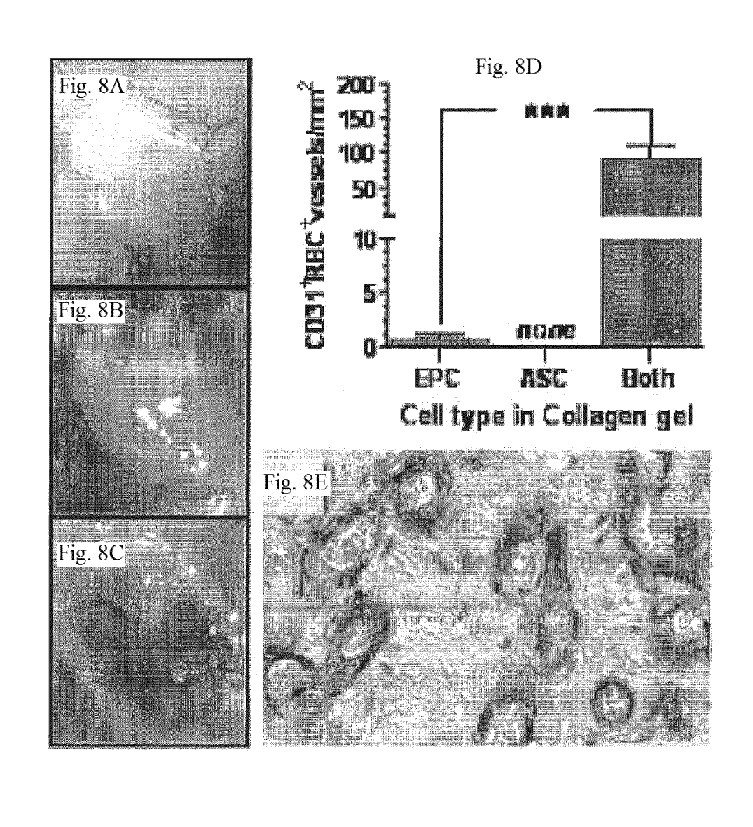

To extend the in vitro finding that ASC promote endothelial cord formation and stability, an in vivo model was employed by embedding ASC together with EPC in 3D collagen gels, followed by their implantation subcutaneously into NOD/SCID mice for fourteen days. Gross evaluation of implants, following the fourteen day period, showed an obvious difference between the appearance of gels containing ASC or EPC alone (white, minimally attached to adjacent host tissue) and gels containing their mixture, which were routinely blood-filled and tightly connected to the mouse abdominal wall (FIGS. 8a-c; (a) EC, (b) ASC, or (c) a mixture (4:1) of EC and ASC). Panel d shows a graphical representation of the frequency of functional, multilayered vessels was formed in implants containing the EPC, ASC, or a mixture of both cell types. Panel e shows a micrographic image of a thin section from an implant that was probed with a smooth muscle α-actin and stained with eosin showing erythrocyte-containing vessels. Analysis of vascular density in the implants, performed by probing sections for human CD31, revealed that gels carrying only EC showed many fewer vessels than gels implanted carrying both EC and ASC (FIG. 8d) IC staining revealed that vessels in the EC+ASC group consistently demonstrated: (1) multilayered structure, with an inner layer formed by the input EC and an outer layer by ASC; (2) both layers were comprised of donor (human) cells; and (3) red blood cells in the lumen, confirming that the chimeric vasculature had established functional anastomoses to adjacent host (mouse) vessels (FIG. 8c). Therefore, the robust ability of ASCs to support vessel network assembly in vivo as well as in vitro was shown. Furthermore, these networks were found to persist for up to 6 weeks (the length of the experiments).

### Example 6

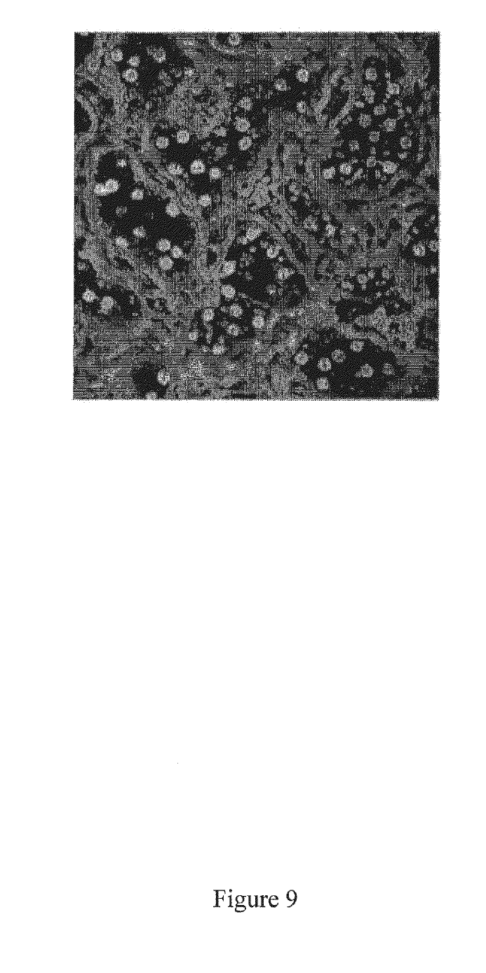

To permit serial assessment of vascular network assembly over time in vivo, intravital imaging was employed using imaging of circulating dextran to identify patent vessels; this imagery also permitted evaluation of blood flow rates as well as localization of differentially labeled input cells (e.g, GFP or dsRed labeled, in the context of blue dextran blood pool imaging). FIG. 9 illustrates imaging the vasculature of a kidney. In this examination, anesthesized mice were injected with Hoechst (blue) to stain nuclei and rhodamine dextran (red) to label blood vessels. The shadows within the red vessels indicate blood flow. Such images undergo 3-dimensional reconstruction and voxel-based image analysis. After subtracting background, volume renderings of the sample are generated using Voxx for visual analysis and to permit computation of fractional vessel (as well as parenchymal cell) volumes.

### Example 7

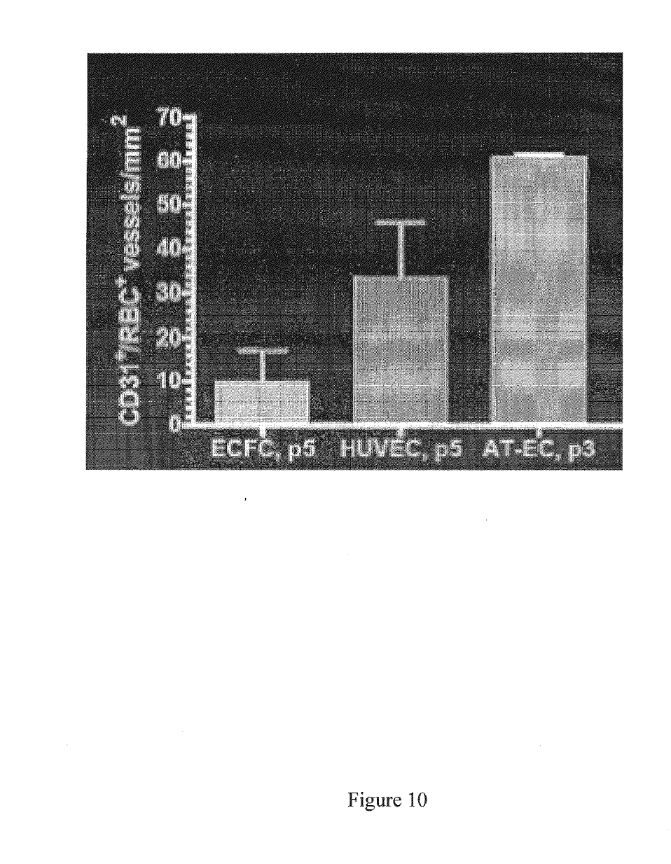

In order to determine whether the approach of vascular network formation by ASC and EC could be employed with cells that could be derived in a fully autologous approach, implants designed exactly as above, except using endothelial cells obtained from adipose microvasculature, CD 144+ endothelial cells were sorted from fresh preparations of adipose tissue. Further, the behavior of implants incorporating endothelial cells from human umbilical vein wall (HUVECs) were evaluated. Both sources of ECs demonstrated high frequency formation of chimeric RBC-filled vessels in the implants (FIG. 10). Multiple sources of ECs, including a potentially autologous source from adipose tissues (AT-EC), support vessel formation in the gel implants. Human ASCs were combined in collagen gels with highly proliferative human ECs (ECFC) and human umbilical vein ECs (HUVEC) or human AT-ECs. At 2 weeks, the gels were removed and analyzed as above.

### Example 8

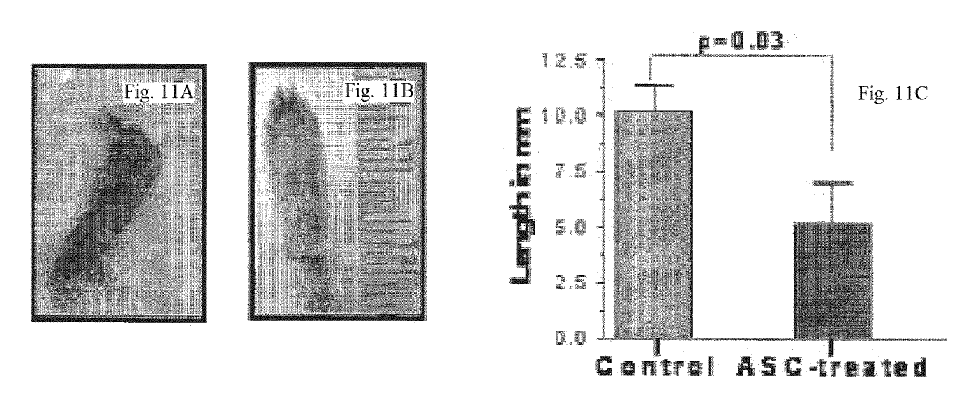

Given the secretion of angiogenic factors by ASC, the angiogenic potential of hASC in skeletal muscle was examined in vive was using two ASC delivery approaches, including: local (intramuscular), and systemic (IV, tail-vein) injections. In the first approach, immunodeficient NOD/SCID mice underwent unilateral femoral artery ligation and received intramuscular injection of either 4×10⁵ human ASCs per hindlimb or media into the m. quadriceps, m. gastrocnemius and m. tibialis anterior of the ischemic hindlimb on the subsequent day (5 injections of 100 μl total). By day 10 of the study (FIG. 11), mice receiving ASC injections had a remarkably reduced extent of foot and toe necrosis (p=0.03). Panels A and B show representative photos of limb necrosis in media-injected (A) and in ASC-injected (B) mice.

### Example 9

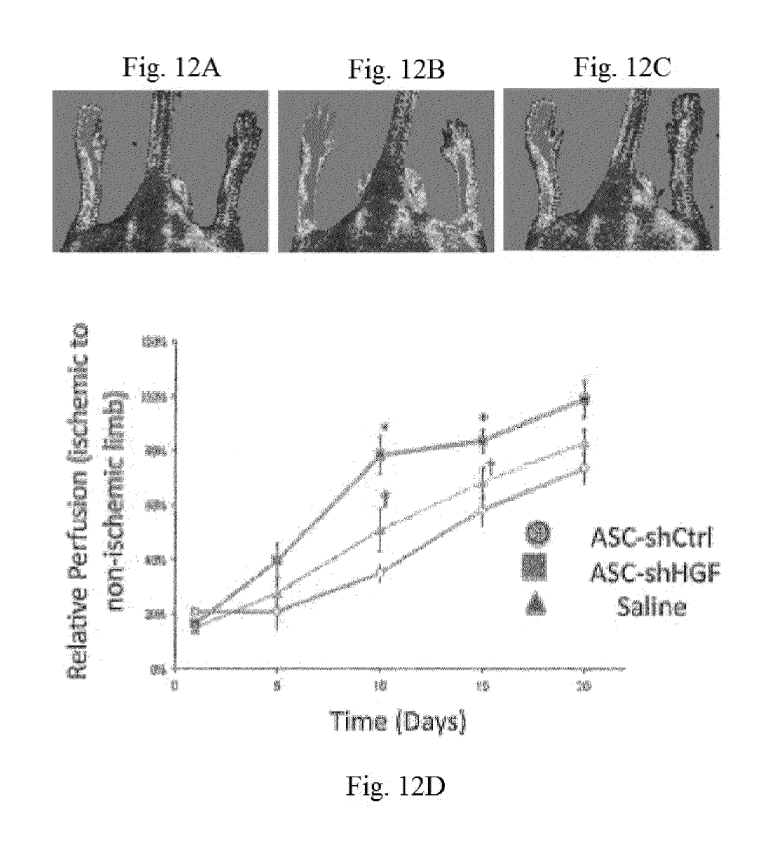

Based on the known anti-apoptotic effect of HGF, we assessed whether HGF played a critical role in the tissue-preserving effects of ASCs by evaluating the effects of ASC modified either by a lentiviral vector expressing an sh-RNA against HGF (shHGF), or by a control null vector (shCtrl) (FIG. 12). Representative blood flow images of mouse hindlimbs treated with (A) saline, (B) ASCs transfected with siControl (ASC-shCtrl) and (C) ASCs transfected with siHGF. Panel D shows relative perfusion (ischemic to nonischemic limb) over time in mice treated with saline, ASC-shCrtrl and ASC-siHGF, as indicated. Silencing of HGF in the ASCs significantly abrogated their beneficial activity, highlighting HGF as a key paracrine effector of ASCs.

### Example 10

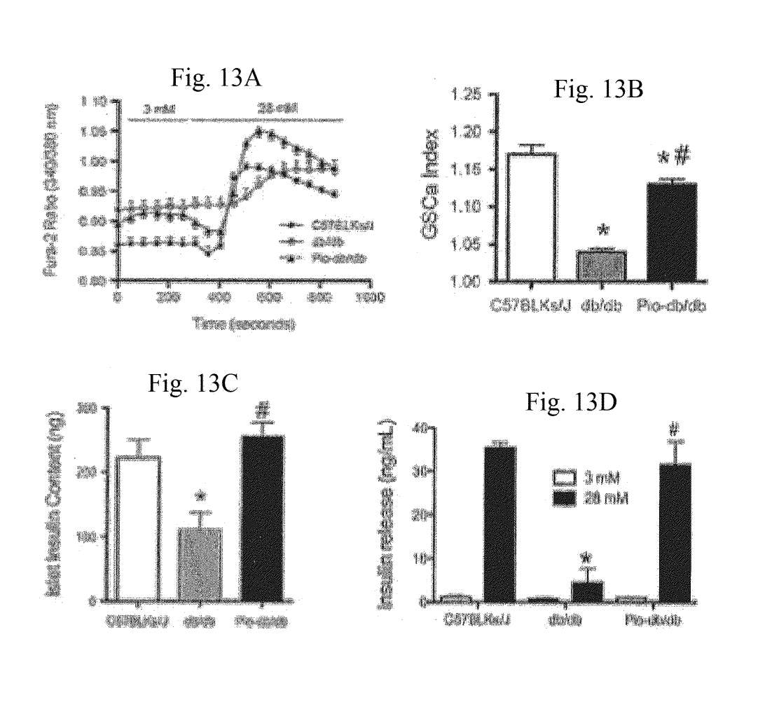

Explanted islet function are characterized in vitro by the response to glucose exposure, resulting in a burst of cytoplasmic Ca²⁺, evaluated by ratiometric fura-2 fluorescence imaging; this leads to insulin release into the media, also measurable as a downstream functional index. These assays are illustrated in FIG. 13 where obese diabetic db/db mice were treated with either saline control (db/db) or pioglitazone (Pio db/db) for 6 weeks by ip. Islets were isolated for functional analysis and compared to lean, normoglycemic mice from the background strain (C57BLKs/J). (A) Glucose-stimulated calcium response of islets from the 3 groups. (B) Peak glucose-stimulated calcium response in the 3 groups. (C) Mean islet insulin content in islets from the normoglycemic control, control treated db/db mice (db/db), and Pio-treated db/db mice (Pio-db/db). (D) Islets were isolated and exposed to 3 mM and then 28 mM glucose for 1 hour.

### Example 11

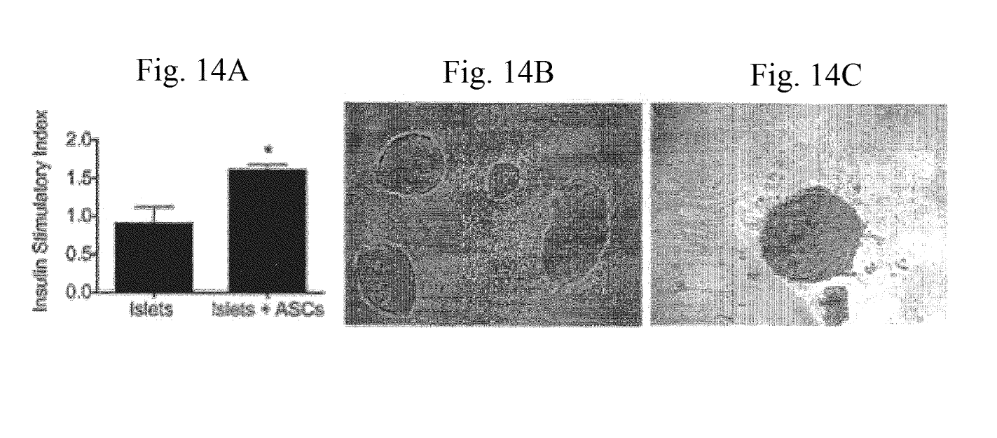

Islets from normoglycemic C57BL6/J mice were isolated and cultured either alone or on top of an ASC monolayer for 7 days. To assess islet function in both cases, glucose stimulated insulin secretion was measured at the end of experiment. Glucose-stimulated insulin secretion after prolonged culture was significantly higher by islets cultured with ASCs (FIG. 14). Panel A shows Insulin Stimulatory Index, which is a ratio of insulin secreted at high (25 mM) and low (2.5 mM) glucose (*, p<0.05). Panel B shows the morphology of islets cultured for 7 days on ASC monolayers. Panel C shows islets only cultured for 7 days. Further, the morphology of islets cultured with ASCs was significantly preserved compared to islets cultured alone. Panels B and C show breakdown of the islet capsule after 7 days in islets cultured alone, reflective of de-differentiation that has been described as an epithelial-mesenchymal transition; this was inhibited by culture either on or above ASC monolayers.

### Example 12

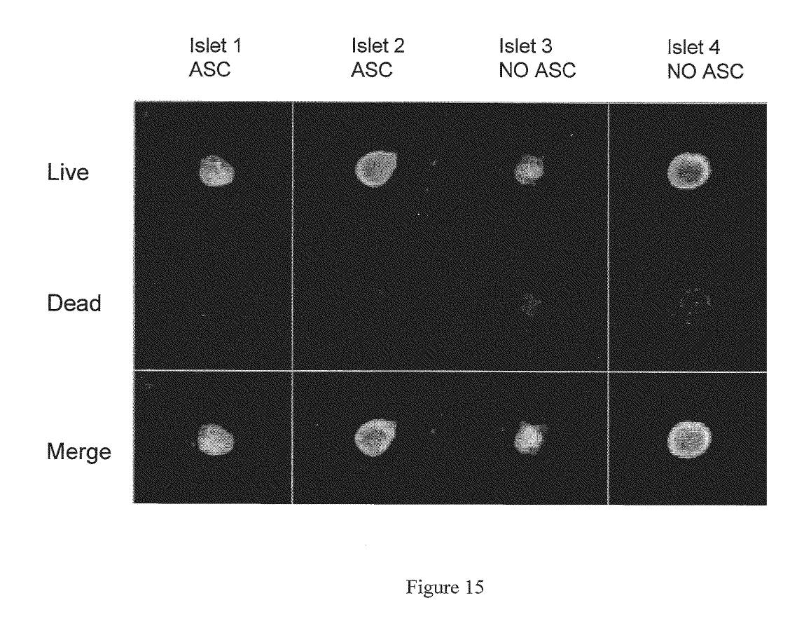

Islets from normoglycemic C57BLK6/J mice were isolated and cultured either alone or with an ASC monolayer for 7 days, then stained with green Calcein AM (green) or red Propidium Iodide (PI)(red) to detect live and dead cells, respectively. FIG. 15 shows two islets cultured alone and two cultured above ASC in a transwell, illustrating a marked repression of islet cell death by substances secreted by ASC; quantitation of discrete PI-stained nuclei per islet cross-section at the equator of the islets revealed reduced dead cells in islets with ASC, to 21% of the number identified in control islets.

### Example 13

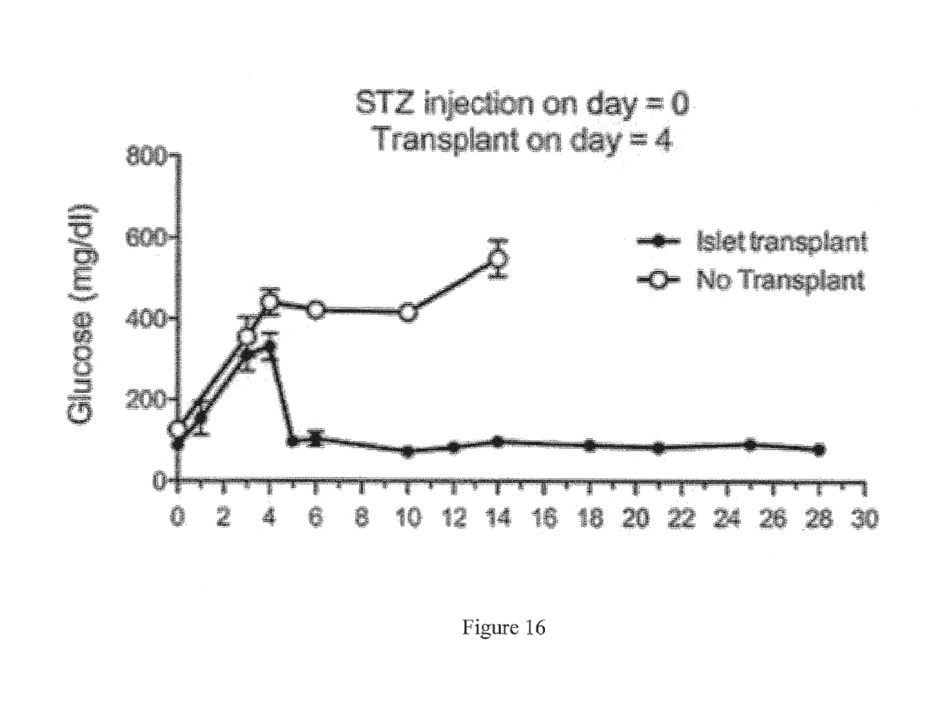

To confirm the feasibility of induction of Type 1 diabetes in immunocompromised (NOD-SCID) mice as a model in which to evaluate islet implant function and stability, these mice were subjected to streptozotocin administration to induce failure of endogenous islets, and monitoring of their glucose levels (FIG. 16). Heterotypic transplantation of pancreatic islets resulted in the restoration of normoglycemic state in type 1 diabetic mice (filled circles). Mice without transplants (open circles) demonstrated persistent hyperglycemia.

### Example 14

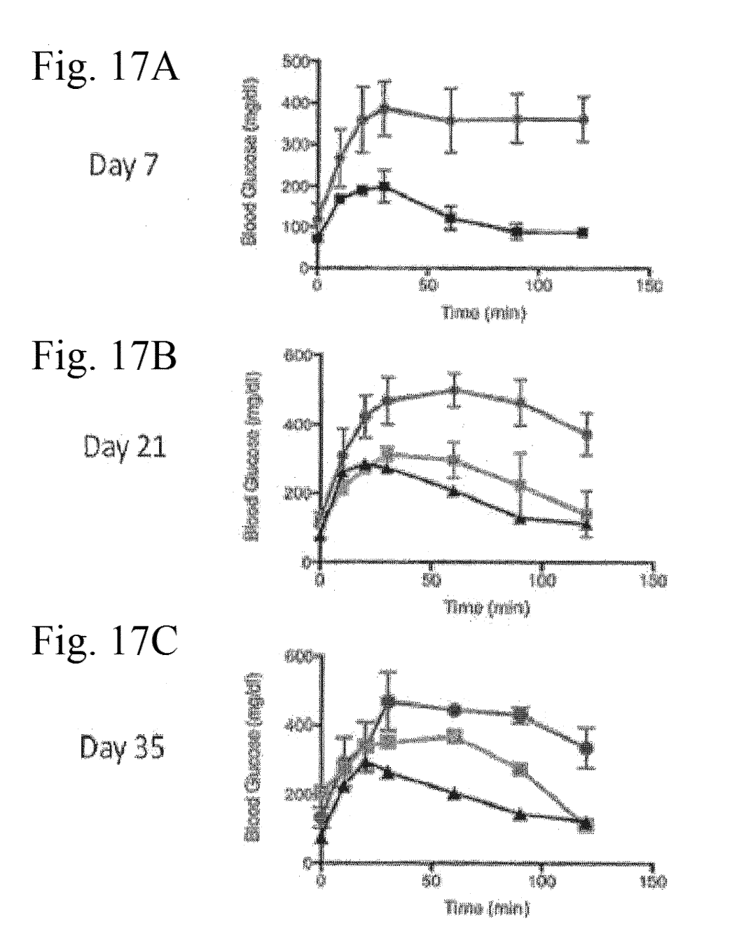

Streptozotocin (STZ) is a nitrosurea compound that is preferentially taken up by the GLUT2 transporter in β cells and acts as an alkylating agent. STZ administered in multiple low doses (MLD-STZ) over 5 days has been shown to reliably induce insulitis and a Type 1 diabetes phenotype within 1-2 weeks via a T cell mediated streptozotocin-induced diabetes. NOD-SCID mice were treated in this experiment with STZ at a dose of 55 mg/kg per day for 5 days (FIG. 17A). Glucose intolerance was demonstrated following STZ injection (FIGS. 17B and C) and ASCs were delivered systemically by tail vein injection ten days after initiation of STZ injection. Glucose tolerance was increased in type 1 diabetic mice (blue) following treatment with ASCs on Day 10 (red). For comparison, serum glucose levels in normal mice (black) are also shown. Glucose homeostasis was assessed 11, and 25 days after ASC injection (Day 21 and 35 post initiation of STZ). Streptozotocin-treated mice treated with ASCs in these experiments showed improved glucose tolerance.

### Example 15

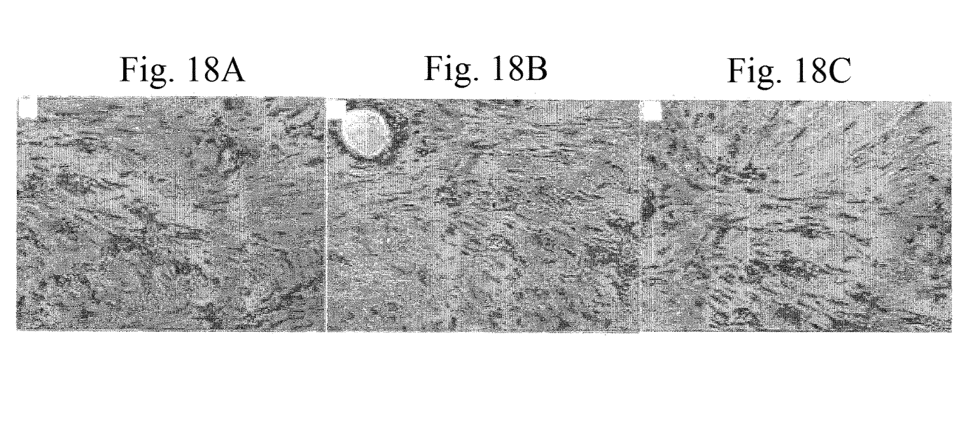

The histology of collagen matrix constructs containing islets admixed with ASC and EC implanted into NOD SCID mice was evaluated. These implants (FIG. 18) showed that functional vessels (Panels A, Hematoxylin and eosin stained sections; and B, Immunohistochemical detection of human CD31-expressing vessels) were adjacent to insulin-staining cells (Panel C, porcine insulin), confirming the survival of 3 cells in this construct for 2 weeks.

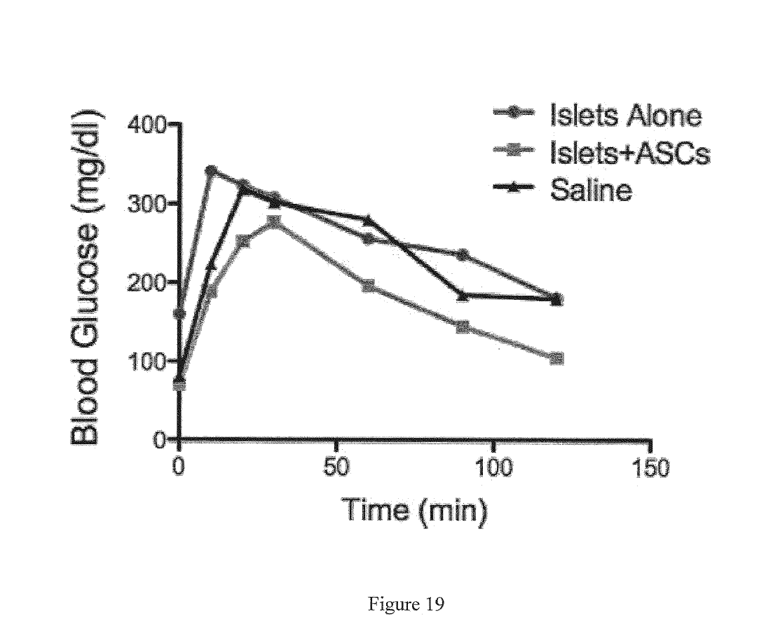

STZ, when given as a single high dose, results in 3 cell ablation and hyperglycemia. Eight week-old NOD-SCID mice were treated with one injection of STZ at a dose of 150 mg/kg. Two days post-STZ administration, diabetes was documented in treated mice, and either 250 islets alone, 250 islets combined with 2×10⁵ ASCs, or saline carrier (control; no islets) were transplanted under the kidney capsule of recipient mice. Blood glucose tolerance was assessed 16 days post transplantation by challenging with glucose and measuring blood levels over time. Mice co-transplanted with a combination of ASCs and islets exhibited improved glucose tolerance as compared to saline controls (FIG. 19).

### Example 16

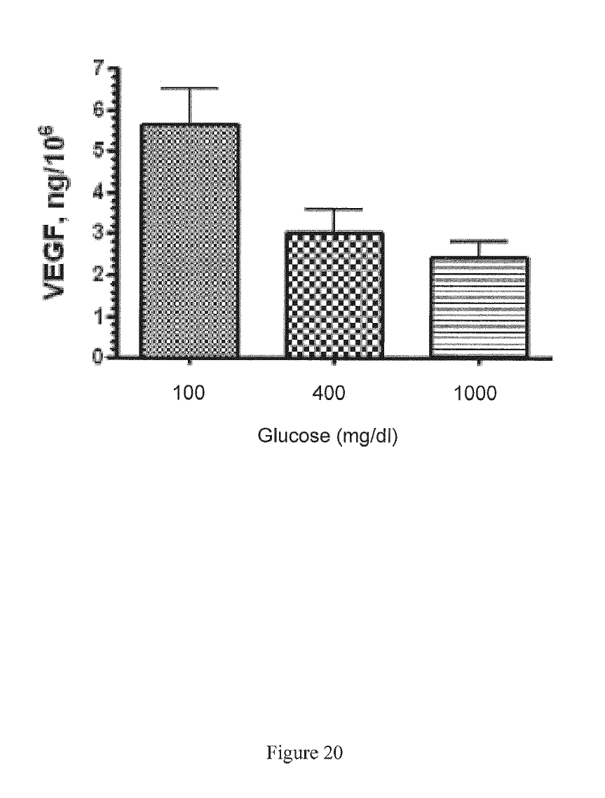

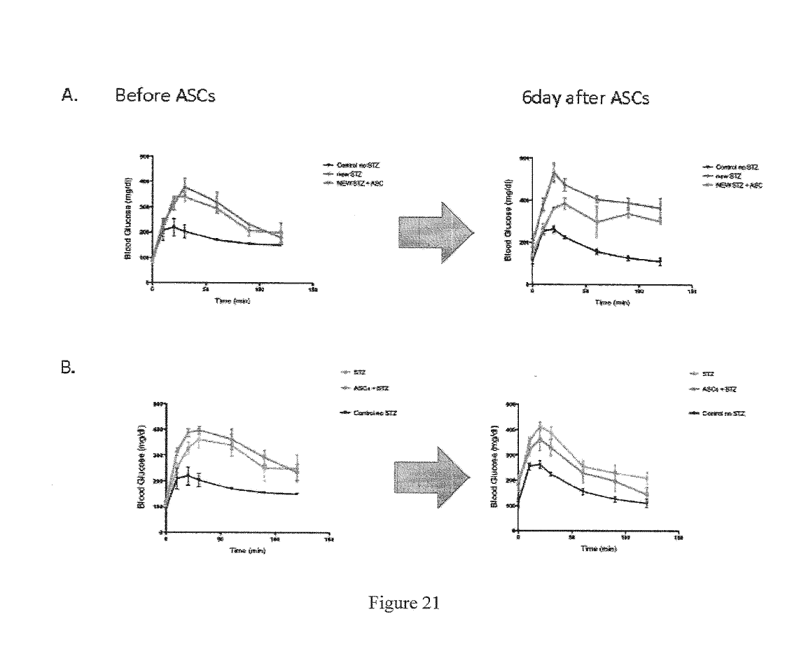

VEGF secretion by human ASC is markedly repressed by diabetic levels of glucose. The effects of glucose concentration in culture medium on the expression of VEGF by ASC were assessed. ASCs were cultured for seventy-two hours in the presence of physiologic glucose (100 mg/dl, in typical clinical units), or with diabetic or extreme glucose levels (400, and 1000 mg/dl respectively). VEGF secretion into the medium, expressed as ng/10⁶ cells/24 hours was attenuated by more than 50% under hyperglycemic conditions (FIG. 20). In this assay, two different groups of 8 week old NOD-SCID mice were treated with streptozotocin to induce β cell damage, which is expected to induce alterations in glucose homeostasis. To determine the effect of ASC's on the prevention of β cell damage, groups of 3-4 mice were injected with either 2 million ASCs or 2 million normal human dermal fibroblasts, (which served as a negative control). Glucose homeostasis was assessed 6 days after tail vein injection of ASCs or control cells by intraperitoneal glucose tolerance test, where 1 gram/kg of glucose was injected intraperitoneally followed by assessment of blood glucose values at time 0, 15, 30, 60, and 120 minutes (FIG. 21). Results showed that mice injected with ASC's have improved glucose homeostasis, suggesting that ASCs can protect again STZ-induced β cell damage.

### Example 17

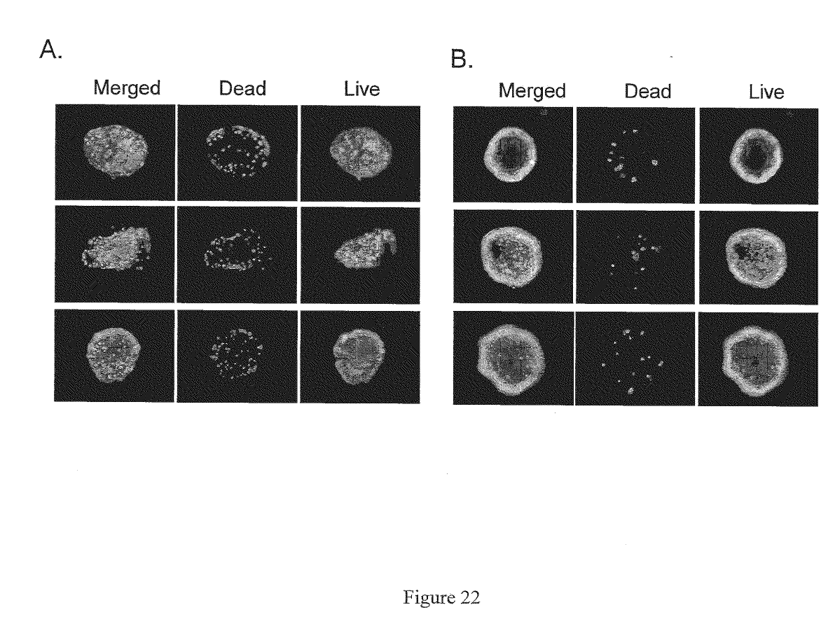

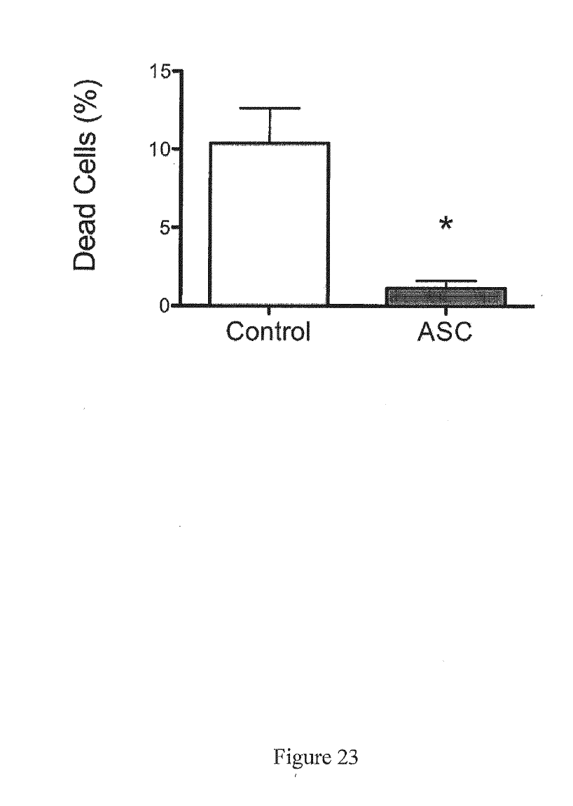

To determine the effect of ASC on viability of islet cells, islets from mice were cultured with or without ASC for six days, and then assayed for effect. In the examination of the effect of ASC, live/dead staining was performed and the percentage of live and dead cells were quantitated (green staining indicates live cells and red indicates dead cells). As determined from this analysis, the level of dead cells in the samples with ASC (FIG. 22B) was significantly lower than that with no ASC present (FIG. 22A). The percentage of dead cells were quantitated for multiple islets and are shown on FIG. 23. Upon analysis, the average percentage of death for the islets exposed to ASC was significantly reduced. In addition, glucose stimulated insulin secretion assays were also performed. Islets cultured alone were unhealthy and not able to secrete insulin in response to glucose stimulation (data not shown). In contrast, islets cultured with ASCs shows clear increase of insulin secretion. indicates that ASCs have positive effects on islets survival and function following prolonged culture (data not shown).

### Example 18

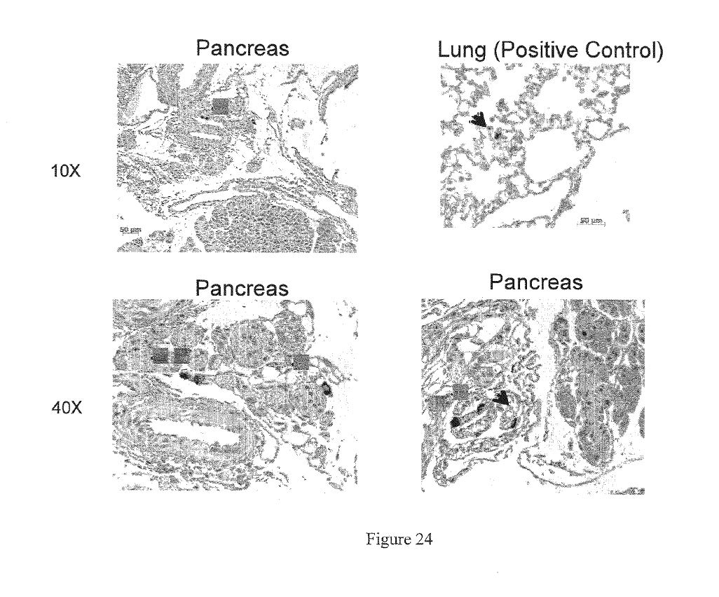

To determine whether the ASC introduced into mice migrated to the kidney, a tail vein injection of ASC was conducted followed up by visualization of the ASC through staining. Immunodeficient NOD-SCID mice were first treated with STZ at a level of 40 mg/kg for the first 4 days, as specified to produce type 1 diabetic mouse model. At day 10, GFP-labeled ASC were injected (2×10⁶ ASC) into a fraction of the diabetic mice via the tail vein. On day 13, lung and pancreas samples were harvested from the mice, stained with anti-GFP antibody, and visualized under microscopy (FIG. 24). The arrows included in the figure direct the attention to the staining by the anti-GFP antibody.

### Example 19

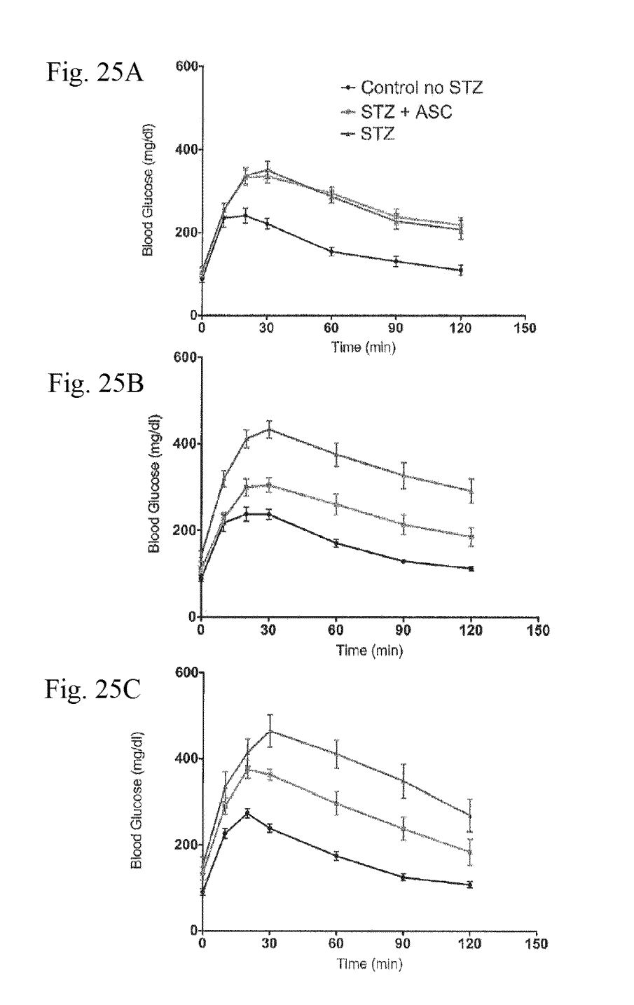

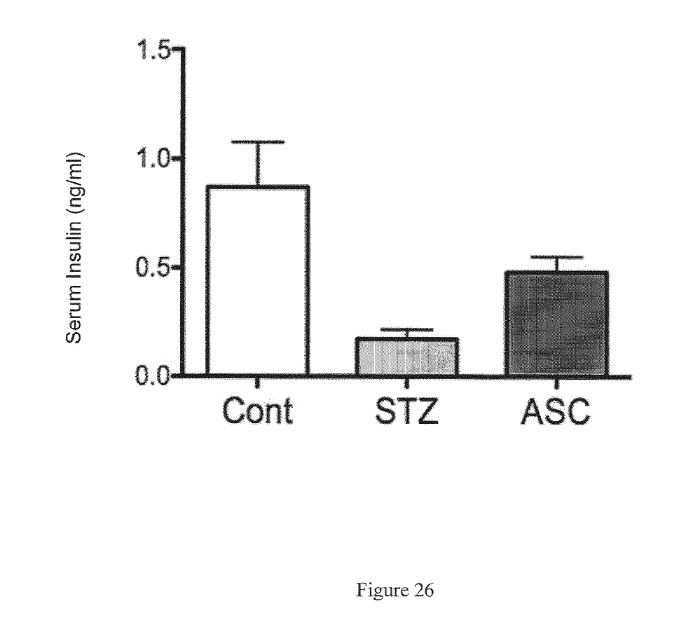

To determine the effect of ASC on glucose homeostasis in a diabetic mouse model, the same tail STZ induction (45 mg/kg used instead of the previous 40 mg/kg) and tail vein injection of ASC as described in Example 18 was performed. For these mice, a glucose tolerance test was performed (1 g/kg glucose injected intraperitoneally) at 7 days (prior to the ASC administration at day 10), as well as at 7 days and 25 days post injection of the ASC (day 17 and 35 respectively). Serum at pancreata were harvested for analysis at the three time points (Pre-ASC injection, 7 days post injection, and 25 days post injection). IP glucose tolerance test results are shown in FIGS. 25A-C (A=pre-ASC injection; B=7 days post-ASC injection; and C=25 days post-ASC injection). Serum insulin levels were also determined for the mice at 7-10 days post ASC administration. For this analysis, serum was collected 30 minutes alter glucose injection and serum insulin levels were measured by ELISA. *p<0.05 compared to control group; p<0.05 compared to STZ group (FIG. 26).

### Example 20

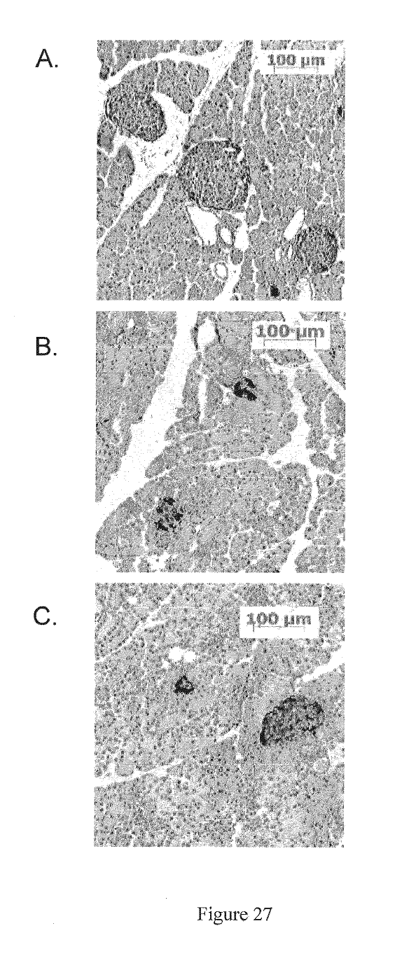

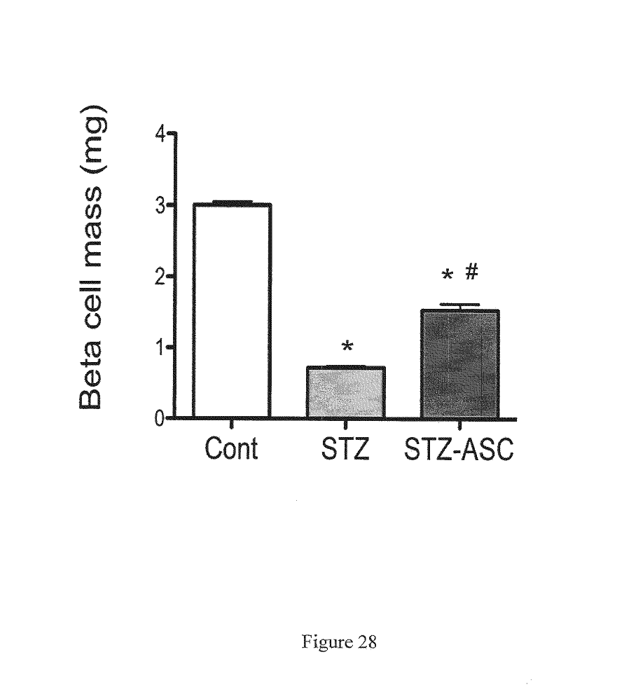

To determine whether ASCs improve beta cell mass in STZ-treated NOD-SCID mice, pancreata were collected 7-10 days post-ASC injection. These collected samples were stained with an insulin-specific antibody to visualize insulin, and beta cell mass was quantitated (FIGS. 27A-C; A=Control, B=STZ only, and C=STZ and ASC). *p<0.05 compared to control group; p<0.05 compared to STZ group. Quantification of Beta cell mass levels in mg is shown in FIG. 28.

### Example 21

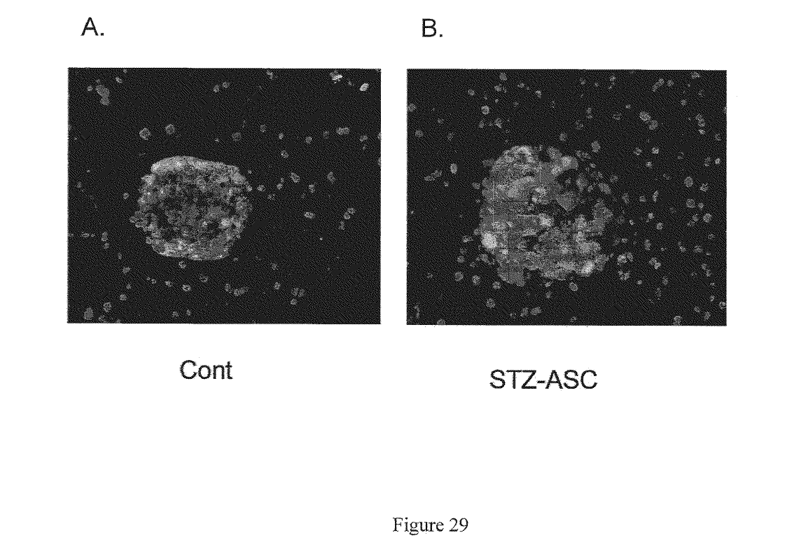

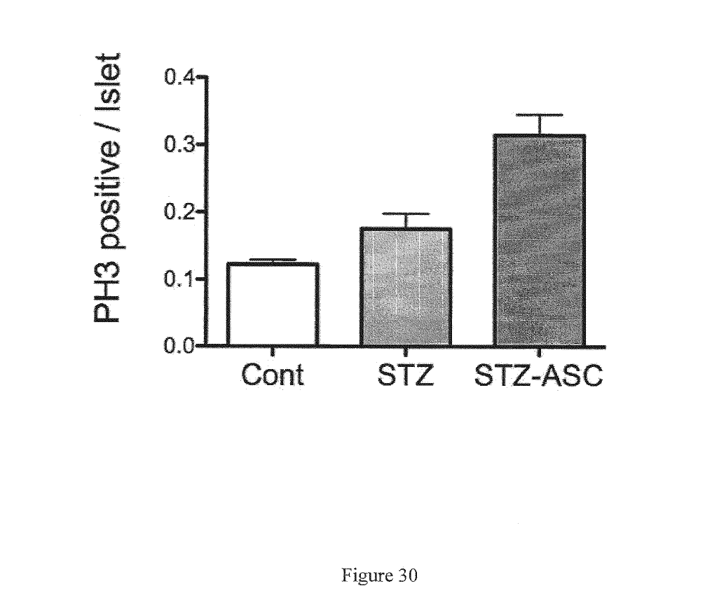

Since the level of Beta cell mass is a balance between cell proliferation and cell death, the level of cell proliferation as affected by ASC was determined. To determine the level of proliferation, an antibody for the proliferation marker Phospho histone 3 (PH3) was used to stain pancreas sections. Under this analysis, all positive nuclei were counted on the islet, but not those outside of the islet. Immunofluorescence visualization of Islets (control or STZ-ASC) stained with anti-insulin, anti-PH3. and DAPI are depicted in FIGS. 29A and B (A=control, B=STZ with ASC). A graphical representation of the results of the PH3 staining/islet are shown in FIG. 30. Proliferation was significantly increased in STZ-ASC treated mice. *p<0.05 compared to control and mice treated with STZ alone.

While various embodiments of cell-based compositions comprising at least one stem cell and methods for using the same have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure. Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of stops may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method for treating a patient with an insulin-related disorder, the method comprising the step of:
administering a cell-based composition to a patient with an insulin-related disorder to treat the insulin-related disorder, the cell-based composition comprising:
at least one mammalian stem cell previously isolated from the patient,
a biological agent capable of promoting cell growth, the biological agent added to the composition in addition to the at least one mammalian stem cell, and
at least one islet cell, the at least one mammalian stem cell capable of prolonging an effective life of the at least one islet cell.

2. The method of claim 1, wherein the step of administering a cell-based composition comprises administering the cell-based composition comprising at least one mammalian adipose stem cell.

3. The method of claim 1, wherein the step of administering a cell-based composition comprises administering the cell-based composition comprising at least one mammalian adipose stem cell and at least one mammalian endothelial or endothelial progenitor cell.

4. The method of claim 1, wherein the step of administering effectuates vascularization of a tissue of the patient at or near the site of administration of the cell-based composition.

5. The method of claim 1, wherein the step of administering a cell-based composition to a patient comprises an administration selected from the group consisting of intravenous injection, intramuscular injection, subcutaneous injection, retrograde venous injection, arterial injection, and surgical implantation.

6. The method of claim 1, wherein the insulin-related disorder is selected from the group consisting of Type 1 diabetes, Type 2 diabetes, gestational diabetes, pre-diabetes, and impaired glucose tolerance.

7. The method of claim 1, wherein the biological agent is selected from the group consisting of a hepatocyte growth factor, an insulin-like growth factor, a fibroblast growth factor, a vascular endothelial growth factor, an anti-apoptotic agent, and a pro-angiogenic agent.

8. The method of claim 1, wherein the at least one mammalian stem cell comprises a plurality of mammalian adipose tissue-derived stem cells, wherein said plurality of mammalian adipose tissue-derived stem cells are preconditioned using the biological agent prior to the step of administering.

9. The method of claim 1, wherein the administration step is performed to treat the insulin-based disorder by promoting production of insulin within the patient.

10. The method of claim 1, wherein the administration step is performed to treat the insulin-based disorder by reducing a rate of peripheral insulin resistance within the patient.

11. The method of claim 1, wherein the administration step is performed to treat the insulin-based disorder by reducing a rate of β-cell dysfunction within the patient.

12. The method of claim 1, wherein the administration step is performed to treat the insulin-based disorder by increasing the patient's glucose tolerance.

13. The method of claim 1, wherein the cell-based composition further comprises at least one endothelial cell, and wherein a ratio of the at least one mammalian stem cell to the at least one endothelial cell is selected from the group consisting of at least about 8 to about 1, about 4 to about 1, about 2 to about 1, about 1 to about 1, about 1 to about 2, about 1 to about 4, and about 1 to at least about 8.

14. A method for treating a patient with an insulin-related disorder, the method comprising the step of:
administering a cell-based composition to a patient with an insulin-related disorder to treat the insulin-related disorder, the cell-based composition comprising:
at least one mammalian adipose stem cell,
a biological agent capable of promoting cell growth, the biological agent added to the composition in addition to the at least one mammalian stem cell, and
at least one islet cell, the at least one mammalian stem cell capable of prolonging an effective life of the at least one islet cell and further capable of effectuating promotion of insulin production within the patient.

15. The method of claim 14, wherein the at least one mammalian stem cell is selected from the group consisting of at least one a CD 10+ mammalian adipose stem cell, at least one a CD 13+ mammalian adipose stem cell, at least one a CD34+ mammalian adipose stem cell, at least one a CD34− mammalian adipose stem cell, at least one a CD45+ mammalian adipose stem cell, at least one a CD45− mammalian adipose stem cell, at least one a CD90+ mammalian adipose stem cell, at least one a CD90− mammalian adipose stem cell, at least one a CD140a+ mammalian adipose stem cell, at least one a CD 140a− mammalian adipose stem cell, at least one a CD140b+ mammalian adipose stem cell, and at least one a CD 140b− mammalian adipose stem cell.

16. The method of claim 14, wherein the cell-based composition further comprises at least one endothelial cell.

17. A method for treating a patient with an insulin-related disorder, the method comprising the step of:
administering a cell-based composition to a patient with an insulin-related disorder to treat the insulin-related disorder, the cell-based composition comprising at least one mammalian adipose stem cell capable of effectuating promotion of insulin production within the patient.

18. The method of claim 17, wherein the at least one mammalian adipose stem cell of the cell-based composition administered to the patient was previously isolated from the patient.

19. The method of claim 17, wherein the cell-based composition further comprises a biological agent capable of promoting cell growth.

20. The method of claim 17, wherein the step of administering a cell-based composition comprises administering the cell-based composition comprising at least one mammalian adipose stem cell and at least one mammalian endothelial or endothelial progenitor stem cell.
